(19) United States
(12) Patent Application Publication
(10) Pub. No.: US 2023/0296747 A1
(43) Pub. Date: Sep. 21, 2023

(54) CONTACT DEGREE CONFIRMATION APPARATUS AND CONTACT DEGREE CONFIRMATION SYSTEM

(71) Applicant: JVCKENWOOD Corporation, Yokohama-shi (JP)

(72) Inventor: Fumiaki MIYAKE, Yokohama-shi (JP)

(21) Appl. No.: 18/201,642

(22) Filed: May 24, 2023

Related U.S. Application Data
(63) Continuation of application No. PCT/JP2021/027553, filed on Jul. 26, 2021.

(30) Foreign Application Priority Data
Dec. 18, 2020 (JP) 2020-210294

(51) Int. Cl. G01S 11/06; H04B 17/318; G16H 50/80

(52) U.S. Cl. CPC G01S 11/06; H04B 17/318; G16H 50/80

(57) ABSTRACT

A user terminal has reception directivity, and includes a beacon reception unit that receives a beacon signal transmitted by anther user terminal, a received signal strength indicator measurement unit that measures a received signal strength indicator of the received beacon signal, and a contact confirmation unit that evaluates the degree of contact with the other user terminal based on a total value of a received signal strength indicator measured in a predetermined period. The beacon reception unit is worn on a predetermined position of a person wearing the user terminal to have reception directivity corresponding to the direction in which the person wearing the user terminal faces.

### CROSS REFERENCE TO RELATED APPLICATION

This application is based upon and claims the benefit of priority from Japanese patent application No. 2020-210294, filed on Dec. 18, 2020, and International application No. PCT/JP2021/027553, filed on Jul. 26, 2021, the disclosures of which are incorporated herein in its entirety by reference.

### BACKGROUND

The present invention relates to a contact degree confirmation apparatus and a contact degree confirmation system.

With the spread of COVID-19, there is a need for a technology for determining the risk of acquiring an infectious disease. For example, as a related technology, Japanese Unexamined Patent Application Publication No. 2015-161987 describes a technology for determining the risk of infection from a travel route of the user.

With an infectious disease such as COVID-19 that could be transmitted by droplet infection, the risk increases by coming into contact with an infected person at close range. For this reason, the Ministry of Health, Labor and Welfare provides a contact confirmation application for confirming contact with an infected person. This contact confirmation application uses the Bluetooth (registered trademark) function of a smartphone to record contact with people and determine the possibility of having contacted an infected person.

### SUMMARY

However, with the contact confirmation application provided by the Ministry of Health, Labor and Welfare (hereinafter sometimes simply referred to as “contact confirmation application”), it may be determined that there may have been contact with an infected person even when the contact actually does not increase the risk of infection, and people may be needlessly forced to stay home or be restricted from taking certain actions. Hence, there is a need for a technology for evaluating contact with higher accuracy.

The present embodiment provides a contact degree confirmation apparatus including: a first signal reception unit that has reception directivity and receives a radio signal transmitted by a signal transmitter; a first received signal strength indicator measurement unit that measures a first received signal strength indicator of the radio signal received by the first signal reception unit; and a contact degree evaluation unit that evaluates the degree of contact with the signal transmitter based on a total value of the first received signal strength indicator measured by the first received signal strength indicator measurement unit in a first predetermined period, in which the first signal reception unit is worn on a predetermined position of a person wearing the contact degree confirmation apparatus to have reception directivity corresponding to a direction in which the person wearing the contact degree confirmation apparatus faces.

The present embodiment provides a contact degree confirmation system including a signal transmitter and a contact degree confirmation apparatus, in which the contact degree confirmation apparatus includes: a first signal reception unit that has reception directivity and receives a radio signal transmitted by the signal transmitter; a first received signal strength indicator measurement unit that measures a first received signal strength indicator of the radio signal received by the first signal reception unit; and a contact degree evaluation unit that evaluates the degree of contact with the signal transmitter based on a total value of the first received signal strength indicator measured by the first received signal strength indicator measurement unit in a first predetermined period, in which the first signal reception unit is worn on a predetermined position of a person wearing the contact degree confirmation apparatus to have reception directivity corresponding to a direction in which the person wearing the contact degree confirmation apparatus faces, or the signal transmitter is worn on a predetermined position of a person wearing the signal transmitter to have transmission directivity corresponding to a direction in which the person wearing the signal transmitter faces.

### BRIEF DESCRIPTION OF THE DRAWINGS

FIG. 1A is a diagram for describing confirmation accuracy of a related technology;

FIG. 1B is a diagram for describing confirmation accuracy of a related technology;

FIG. 1C is a diagram for describing confirmation accuracy of a related technology;

FIG. 1D is a diagram for describing confirmation accuracy of a related technology;

FIG. 1E is a diagram for describing confirmation accuracy of a related technology;

FIG. 2 is a configuration diagram illustrating a configuration example of a contact degree confirmation system according to a first embodiment;

FIG. 3 is a diagram illustrating reception directivity of a reception device according to the first embodiment;

FIG. 4A is a configuration diagram illustrating a configuration example of the reception device according to the first embodiment;

FIG. 4B is a configuration diagram illustrating a configuration example of an information processing unit according to the first embodiment;

FIG. 5 is a flowchart illustrating an operation example of contact information recording processing according to the first embodiment;

FIG. 6 is a flowchart illustrating an operation example of contact confirmation processing according to the first embodiment;

FIG. 7 is a configuration diagram illustrating a configuration example of an information processing unit according to a second embodiment;

FIG. 8 is a flowchart illustrating an operation example of contact confirmation processing according to the second embodiment;

FIG. 9 is a configuration diagram illustrating a configuration example of a contact degree confirmation system according to a third embodiment; and

FIG. 10 is a configuration diagram illustrating a configuration example of a transmission and reception device according to the third embodiment.

### DETAILED DESCRIPTION

Hereinafter, embodiments will be described with reference to the drawings. In the drawings, the same elements are assigned with the same reference numerals and redundant descriptions are omitted as appropriate.

### Consideration of Related Technology

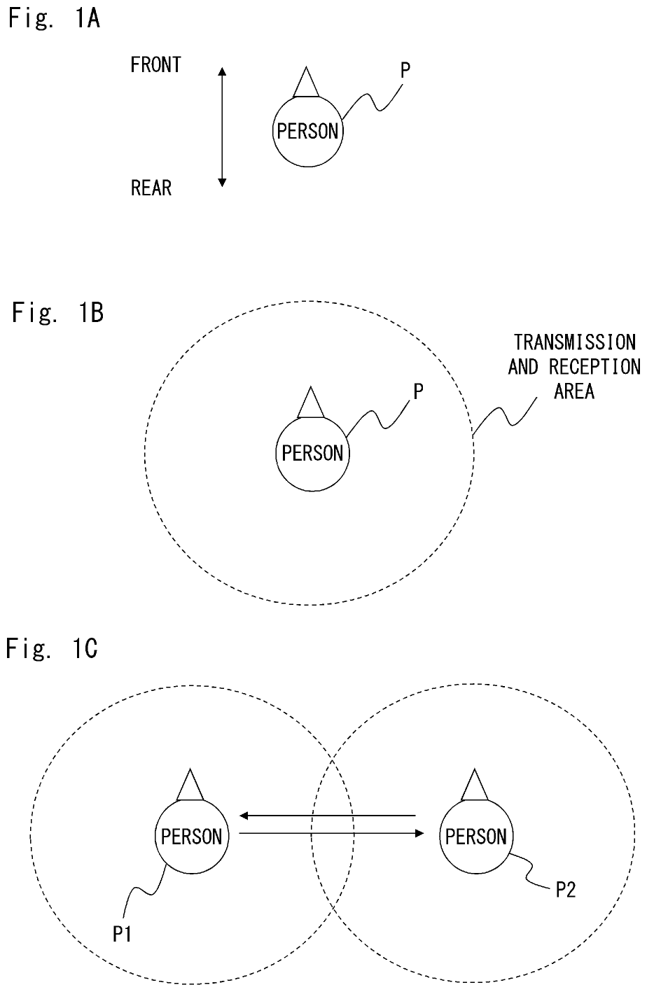

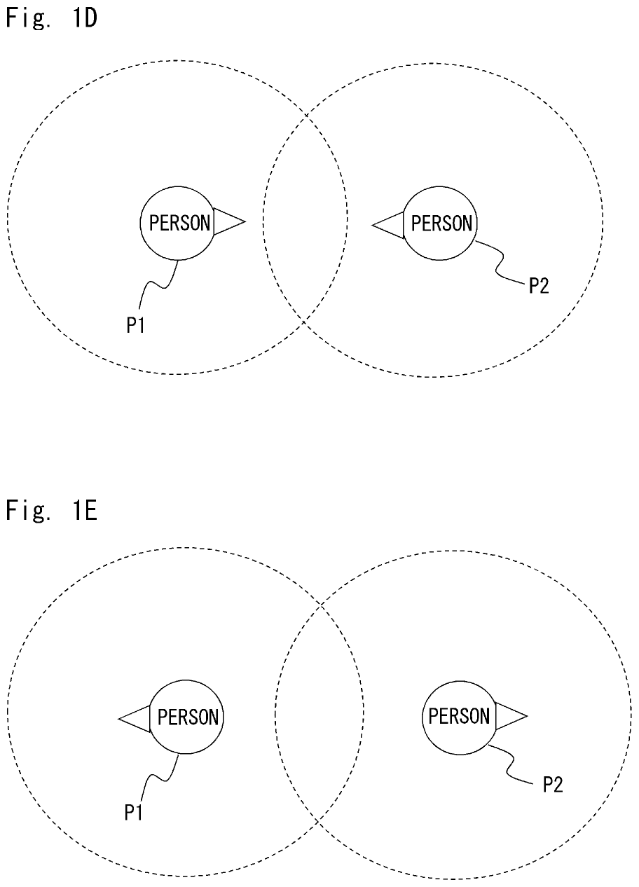

First, the confirmation accuracy of a contact confirmation application before application of the embodiment will be considered with reference to FIGS. 1A to 1E. Here, as shown in FIG. 1A, a model with a person P is defined. FIG. 1A is a model in which the person P is viewed from above, where the circle represents the head of the person P and the triangle represents the nose of the person P. A direction in which the tip end of the triangle nose points is the front direction of the person P's face (front side of face), and a direction opposite to that is the rear direction of the person P's face (side of back of head).

The contact confirmation application operates while being installed in a terminal apparatus such as a smartphone and being carried by the person P. At that time, the contact confirmation application transmits and receives information by radio waves using the Bluetooth function of the terminal apparatus. FIG. 1B indicates a transmission and reception area of Bluetooth radio waves. The transmission and reception area has a circular shape centered on the person P carrying the terminal apparatus. That is, the directivity of Bluetooth of the terminal apparatus in which the contact confirmation application is installed is oriented in almost all directions, and is omnidirectional. This is because the Bluetooth function is not dedicated to use of the contact confirmation application but also enables use of other services.

As illustrated in FIG. 1C, when the person P1 and the person P2 come close to each other and the person P2 enters the radio wave transmission and reception area of the person P1, for example, the contact confirmation applications of the terminal apparatuses of the person P1 and the person P2 perform transmission and reception with each other by radio waves, acquire the received signal strength indicator of the radio waves and the transmitted encrypted information, and record them with time information. Whether or not there has been contact between the person P1 and the person P2 is confirmed by the recorded information.

However, the contact confirmation application only confirms whether or not there is a person nearby, and does not consider more detailed conditions of the person. For example, in the case of an infectious disease that could be transmitted by droplets from a cough or the like, the risk of infection varies depending on the direction in which a person's face is turned. In the example of FIG. 1C, the person P1 and the person P2 are next to each other facing the same direction, in the example of FIG. 1D, the person P1 and the person P2 face each other, and in FIG. 1E, the person P1 and the person P2 are back to back facing opposite directions. While the physical distance between the person P1 and the person P2 is the same in FIGS. 1C to 1E, the risk of receiving droplets differs among the drawings. That is, the risk of infection is the highest in FIG. 1D where the people are facing each other, and the risk of infection is the lowest in FIG. 1E where the people are facing opposite directions.

As described above, the risk of infection varies depending on the direction that people face, but the contact confirmation application does not consider the direction that people face, and therefore cannot confirm contact including the risk of infection. Against this background, in the following embodiments, in addition to the distance between people, directions that people face are considered and weighted to enable confirmation of contact including the risk of infection.

### First Embodiment

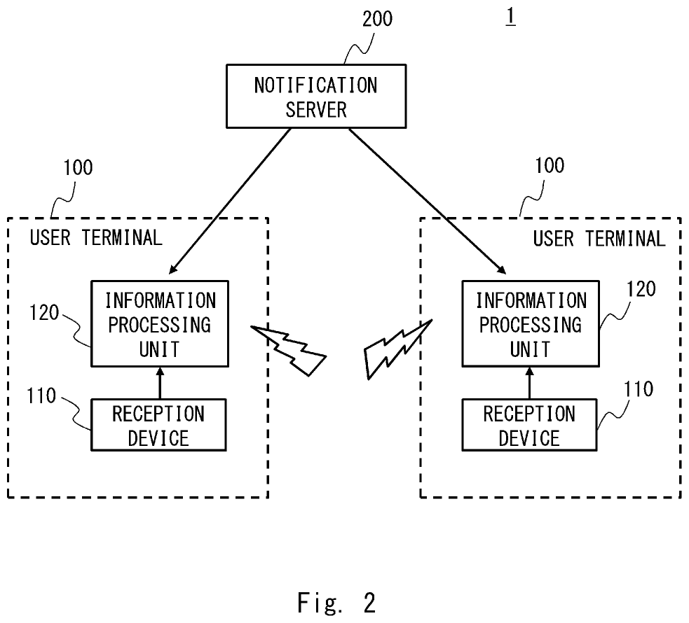

Hereinafter, a first embodiment will be described with reference to the drawings. FIG. 2 illustrates a configuration example of a contact degree confirmation system according to the present embodiment. As illustrated in FIG. 2, a contact degree confirmation system 1 according to the present embodiment includes a plurality of user terminals 100 and a notification server 200. The contact degree confirmation system 1 is a system that confirms the degree of contact with an infected person having caught an infectious disease by exchanging radio signals between the user terminals 100 held (worn) by users. The infectious disease is an infectious disease that could by transmitted mainly by droplets such as COVID-19, for example.

The notification server 200 is a server that manages information regarding infected person, and notifies the plurality of user terminals 100 of infected person information including encrypted identification information of an infected person. The notification server 200 and the user terminal 100 are connected by a network such as the Internet, for example, but may be directly connected by a serial line instead.

The user terminal 100 transmits and receives beacon signals as radio signals to confirm the degree of contact between the user terminals 100. A beacon signal is a signal for measuring the distance between a transmitter and a receiver, and is iBeacon (registered trademark) of Bluetooth Low Energy (BLE), for example. Note that the beacon signal is not limited to Bluetooth, and may be other radio signals such as wireless LAN and the like.

The user terminal 100 includes a reception device 110 and an information processing unit 120, for example. The reception device 110 is a beacon signal receiver, and is a Bluetooth reception device, for example. The information processing unit 120 is a confirmation apparatus that confirms the degree of contact with another user terminal 100 based on information of beacon signals received by the reception device 110 and information notified by the notification server 200, and is also a transmitter that transmits beacon signals. For example, the information processing unit 120 is an information processing apparatus such as a smartphone. Note that the reception device 110 and the information processing unit 120 may be separate apparatuses or may be an integrated apparatus. For example, functions of the information processing unit 120 and the reception device 110 may be included in a smartphone that the user can wear. The reception device 110 and the information processing unit 120 may be connected wirelessly by Bluetooth, wireless LAN, or the like, or may be connected by wire such as a serial line or wired LAN.

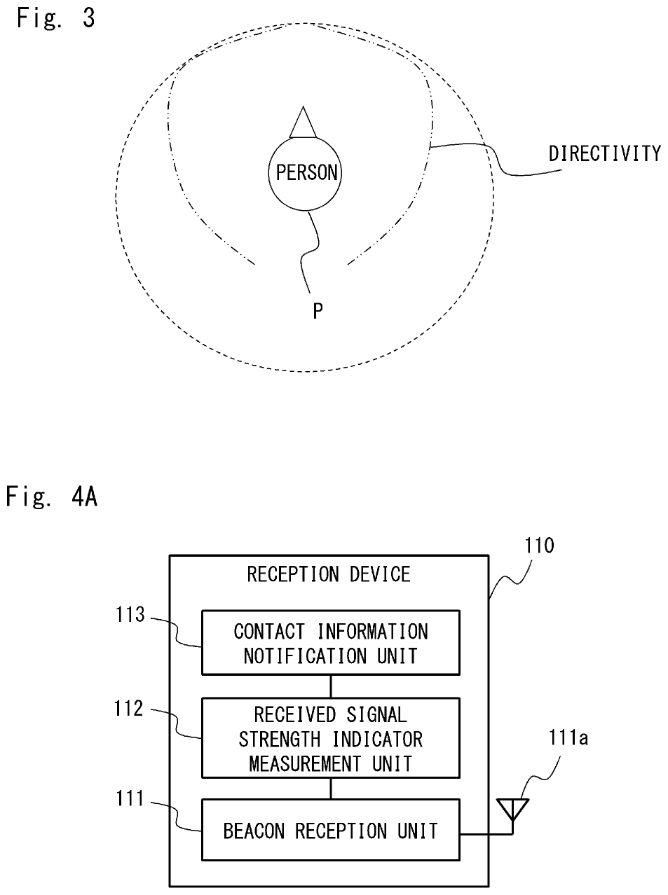

FIG. 3 indicates the reception directivity of radio waves of the reception device 110 according to the present embodiment. As illustrated in FIG. 3, the reception device 110 has reception directivity in a specific direction. That is, the reception characteristic of the reception device 110 has a strong pattern in one direction and a weak pattern in other directions. In the present embodiment, the reception device 110 has reception directivity corresponding to the direction of the face or body of the person wearing the user terminal 100. That is, the reception device 110 is placed (worn) in a predetermined position of the user so that the direction of the user (P)'s face corresponds to (is the same direction as) the reception directivity of the reception device 110. In order to determine the direction in which the user's face is turned, the reception characteristic is set to be strong on the front side of the face and weak at the side of the face or at the back. For example, the reception device 110 may be attached near the face such as on a mask, glasses, or hat, or may be worn on the body such as in a breast pocket. Incidentally, it is preferable that the direction in which the infectious disease is likely to be transmitted to people by droplets and the like correspond to the reception directivity of the reception device 110.

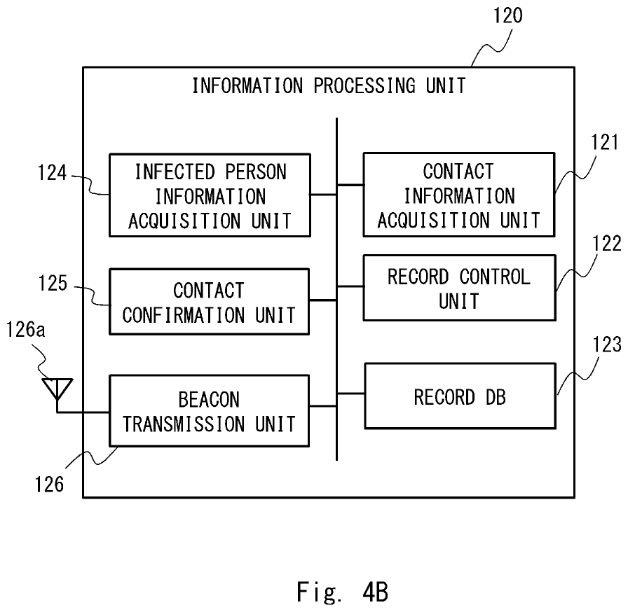

FIG. 4A illustrates a configuration example of the reception device 110 according to the present embodiment. As illustrated in FIG. 4A, the reception device 110 includes a beacon reception unit 111, a received signal strength indicator measurement unit 112, and a contact information notification unit 113.

The beacon reception unit (first beacon reception unit) 111 receives beacon signals wirelessly transmitted from the user terminal 100 which is a nearby signal transmitter according to a communication standard such as BLE. The received beacon signal includes encrypted identification information of the transmission source user. A reception antenna 111a of the beacon reception unit 111 is a directional antenna, and has reception directivity corresponding to the person's direction as described with reference to FIG. 3.

The received signal strength indicator measurement unit (first received signal strength indicator measurement unit) 112 measures the received signal strength indicator (RSSI) (first received signal strength indicator) of the beacon signal received by the beacon reception unit 111. The contact information notification unit 113 notifies the information processing unit 120 of contact information (first contact information) including the encrypted identification information of the user of the transmission source of the beacon signal received from the nearby user terminal 100 and the measured received signal strength indicator of the beacon signal by Bluetooth or the like.

FIG. 4B illustrates a configuration example of the information processing unit 120 according to the present embodiment. As illustrated in FIG. 4B, the information processing unit 120 includes a contact information acquisition unit 121, a record control unit 122, a record DB 123, an infected person information acquisition unit 124, a contact confirmation unit 125, and a beacon transmission unit 126.

The contact information acquisition unit 121 acquires contact information including the encrypted identification information of the user of the transmission source of the beacon signal and the received signal strength indicator from the reception device 110 by Bluetooth or the like. The record control unit 122 performs control such that the acquired contact information is recorded in the record DB 123. The record control unit 122 records the encrypted identification information of the user of the transmission source of the beacon signal and the received signal strength indicator included in the contact information every predetermined period. The record control unit 122 also eliminates the recorded contact information after passage of a certain period. The record DB (first contact information recording unit) 123 records contact information under the control of the record control unit 122. The record DB 123 records the received signal strength indicator together with the received time for each piece of encrypted identification information of the transmission source user.

The infected person information acquisition unit 124 acquires the encrypted identification information of an infected person from the notification server 200 via a network or the like. The contact confirmation unit (contact degree evaluation unit) 125 confirms contact with the user terminal 100 of the infected person based on the received signal strength indicator of the beacon signal. The contact confirmation unit 125 evaluates the degree of contact with the user terminal 100 of the infected person based on the total value of the received signal strength indicator of the beacon signals transmitted from the user terminal 100 of the infected person measured in a predetermined period (first predetermined period) and recorded in the record DB 123. For example, distances calculated from the received signal strength indicator may be weighted, and the degree of contact is evaluated based on the total value of weights.

The beacon transmission unit (signal transmitter) 126 wirelessly transmits beacon signals according to a communication standard such as BLE. The transmitted beacon signal includes encrypted identification information of the transmission source user as in the case of the received beacon signal. In this example, a transmission antenna 126a of the beacon transmission unit 126 is an omnidirectional antenna, and has transmission directivity oriented in all directions.

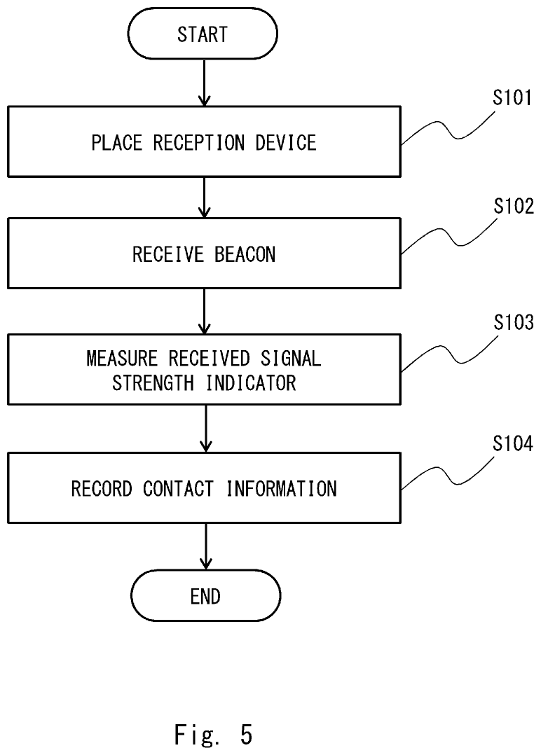

FIG. 5 illustrates an example of contact information recording processing of the user terminal 100 according to the present embodiment. As illustrated in FIG. 5, first, the reception device 110 is placed (S101). As illustrated in FIG. 3, the reception device 110 is mounted on the user's mask or the like, and is placed such that the reception directivity of the reception device 110 corresponds to the direction of the user's face or body.

Subsequently, the user terminal 100 receives a beacon signal (S102). The beacon transmission unit 126 of another user terminal 100 periodically transmits beacon signals including encrypted identification information of the transmission source user. When the other user terminal 100 comes close to the user terminal 100, the beacon reception unit 111 of the reception device 110 receives a beacon signal from the other user terminal 100. The beacon reception unit 111 acquires the encrypted identification information of the transmission source user included in the received beacon signal.

Subsequently, the user terminal 100 measures the received signal strength indicator of the beacon signal (S103). The received signal strength indicator measurement unit 112 of the reception device 110 measures the received signal strength indicator of the beacon signal received from the other user terminal 100. The received signal strength indicator measurement unit 112 measures the received signal strength indicator of a beacon signal every time a beacon signal is periodically received.

Subsequently, the user terminal 100 records contact information based on the beacon signal (S104). The contact information notification unit 113 of the reception device 110 generates contact information from the encrypted identification information of the user of the transmission source of the received beacon signal and the measured received signal strength indicator of the beacon signal and notifies the information processing unit 120 of the generated contact information. The contact information acquisition unit 121 of the information processing unit 120 acquires the notified contact information, and the record control unit 122 records the acquired contact information in the record DB 123. When receiving beacon signals having the same identification information for a predetermined period, the record control unit 122 records the encrypted identification information of the user of the transmission source of the beacon signal and the received signal strength indicator in the record DB 123 together with the received time.

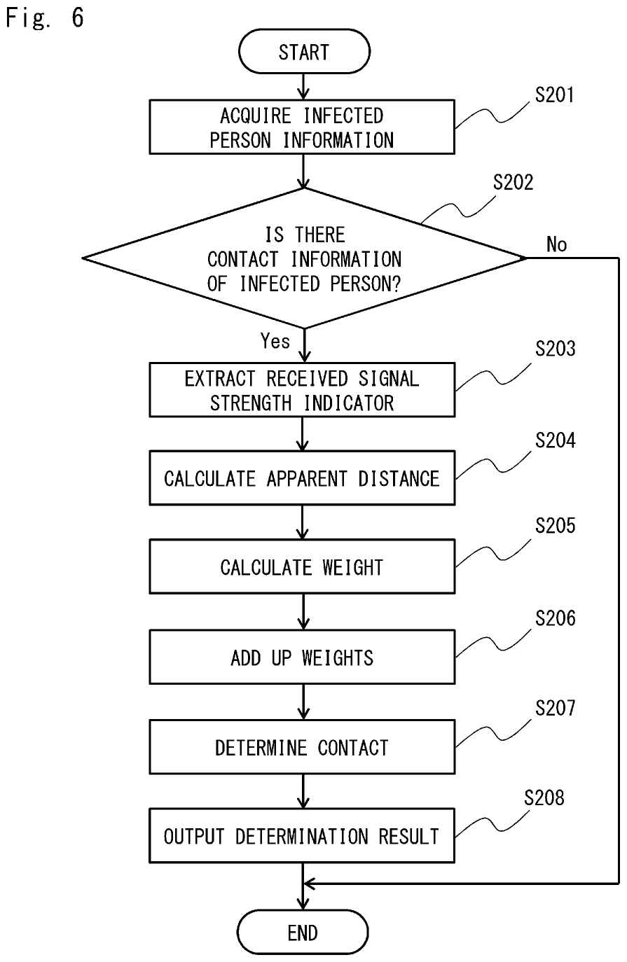

FIG. 6 illustrates an example of contact confirmation processing of the user terminal 100 according to the present embodiment. As illustrated in FIG. 6, first, the user terminal 100 acquires infected person information (S201). When a user having caught an infectious disease (infected person) registers information regarding the infected person in the notification server 200, the notification server 200 notifies the user terminal 100 of encrypted identification information of the infected person, and the infected person information acquisition unit 124 of the user terminal 100 acquires the notified identification information.

Subsequently, the user terminal 100 determines whether or not contact information of the infected person is recorded (S202). The contact confirmation unit 125 of the user terminal checks the acquired encrypted identification information of the infected person against the encrypted identification information of the user of the transmission source of the beacon signal recorded in the record DB 123. For example, a search is performed as to whether or not contact information corresponding to the identification information of the infected person is in the record DB 123 in the past 14 days. If the contact information of the infected person is not recorded (S202/No), the processing is ended.

On the other hand, if the contact information of the infected person is recorded (S202/Yes), the user terminal 100 extracts the received signal strength indicator for confirming contact (S203). The contact confirmation unit 125 refers to the record DB 123, and extracts the received signal strength indicator from every piece of contact information corresponding to the acquired encrypted identification information of the infected person from the contact information of the past 14 days.

Subsequently, the user terminal 100 confirms contact from the received signal strength indicator (S204 to S207). The contact confirmation unit 125 confirms contact with the infected person based on the extracted received signal strength indicator of the infected person. Specifically, the contact confirmation unit 125 calculates the apparent distance based on the received signal strength indicator (S204), calculates weighting of the apparent distance with respect to a predetermined reference distance (S205), and obtains the total of the weighting (S206). Moreover, the contact confirmation unit 125 determines contact by the total value of the weighting (S207).

A specific example of contact confirmation using the received signal strength indicator will be described. The relationship between a distance d between the transmitter and receiver of the beacon signal and the received signal strength indicator of the beacon signal measured by the receiver is represented by the following equation.

d=10\hat{ }((TxPower−RSSI)/20) (1)

In equation (1), TXPower is the received signal strength indicator when the beacon signal is received with the transmitter and receiver separated by 1 m. Here, TXPower is assumed to be −40 dBm as an example.

For example, when the received signal strength indicator RSSI at the time of actual reception is −40 dBm, the distance d is obtained as follows by equation (1).

d=10\hat{ }((−40−(−40)))/20)=10\hat{ }0=1m

Before application of the present embodiment, the reception directivity of radio waves is oriented in all directions, and therefore the RSSI is fixed at −40 dBm regardless of the direction in which the people face. On the other hand, in the present embodiment, the reception directivity of the reception device to be used is oriented toward the front direction of the person's face, and therefore the RSSI varies depending on the direction in which the people face. In order to evaluate the variation depending on the direction, the variation is quantified as a weight with respect to a reference value. Specifically, the distance of the beacon signal calculated from the received signal strength indicator is calculated as the apparent distance from equation (1), and a weighting coefficient (index) is calculated by a reference distance (e.g., 1 m) with respect to the apparent distance. For example, the weight is calculated as (reference distance)/(apparent distance), but other formulae may be used. Moreover, this weighting calculation is performed every time a certain time passes, and the total of weights is obtained within a predetermined period.

For example, in a case where distances obtained from the received signal strength indicator of every minute are weighted and the total value of 10 minutes is to be obtained, if the received signal strength indicator is always −40 dBm, the total value is 10. This value 10 is used as a reference of the total value to determine the degree of contact. Since the total of weights vary depending on the direction of the person's face or body and the time during which the state was maintained, the risk of infection is determined by comparing the total value and the reference value. Tables 1 to 3 below illustrate the received signal strength indicator of the beacon signal and calculation examples of weights for 10 minutes as a guide for contact confirmation in cases where the people are facing different directions.

Table 1 is a calculation example in a case where two people faced each other and maintained this state for 10 minutes. In this case, the weight is fixed at around 1.0 and the total is 9.72, which is close to the reference 10. Hence, it is determined that the possibility of infection is high.

TABLE 1

| | Time lapse (minute) | | | | | | | | | |
|---|---|---|---|---|---|---|---|---|---|---|
| | 1 | 2 | 3 | 4 | 5 | 6 | 7 | 8 | 9 | 10 |
| Received | −41 | −38 | −42 | −38 | −42 | −40 | −41 | −39 | −43 | −40 |
| signal strength | | | | | | | | | | |
| indicator | | | | | | | | | | |
| [dBm] | | | | | | | | | | |
| Apparent | 1.12 | 0.79 | 1.26 | 0.79 | 1.26 | 1.00 | 1.12 | 0.89 | 1.41 | 1.00 |
| distance | | | | | | | | | | |
| Weight | 0.89 | 1.26 | 0.79 | 1.26 | 0.79 | 1.00 | 0.89 | 1.12 | 0.71 | 1.00 |
| | | | | | | | | | Total | 9.72 |

Table 2 is a calculation example in a case where two people faced each other and maintained this state for 5 minutes, and then one person turned to the side. In this case, the weight decreases after 5 minutes, and the total is 5.87. Hence, it is determined that the possibility of infection is low.

TABLE 2

| | Time lapse (minute) | | | | | | | | | |
|---|---|---|---|---|---|---|---|---|---|---|
| | 1 | 2 | 3 | 4 | 5 | 6 | 7 | 8 | 9 | 10 |
| Received | −40 | −37 | −42 | −41 | −40 | −55 | −58 | −60 | −65 | −50 |
| signal strength | | | | | | | | | | |
| indicator | | | | | | | | | | |
| [dBm] | | | | | | | | | | |
| Apparent | 1.00 | 0.71 | 1.26 | 1.12 | 1.00 | 5.62 | 7.94 | 10.00 | 17.78 | 3.16 |
| distance | | | | | | | | | | |
| Weight | 1.00 | 1.41 | 0.79 | 0.89 | 1.00 | 0.18 | 0.13 | 0.10 | 0.06 | 0.32 |
| | | | | | | | | | Total | 5.87 |

Table 3 is a calculation example in a case where two people faced each other for 3 minutes, and then they stood back to back facing opposite directions. In this case, the weight largely decreases after 3 minutes, and the total is 4.15. Hence, it is determined that the risk of infection is even lower.

TABLE 3

| | Time lapse (minute) | | | | | | | | | |
|---|---|---|---|---|---|---|---|---|---|---|
| | 1 | 2 | 3 | 4 | 5 | 6 | 7 | 8 | 9 | 10 |
| Received | −38 | −40 | −35 | −81 | −83 | −75 | −73 | −85 | −70 | −75 |
| signal strength | | | | | | | | | | |
| indicator | | | | | | | | | | |
| [dBm] | | | | | | | | | | |
| Apparent | 0.79 | 1.00 | 0.56 | 112.2 | 141.3 | 56.23 | 44.67 | 177.83 | 31.62 | 56.23 |
| distance | | | | | | | | | | |
| Weight | 1.26 | 1.00 | 1.78 | 0.01 | 0.01 | 0.02 | 0.02 | 0.01 | 0.0 | 0.02 |
| | | | | | | | | | Total | 4.15 |

Subsequently, the user terminal 100 outputs the result of contact determination (S208). The contact confirmation unit 125 gives an alert corresponding to the degree of contact on a display unit or the like of the user terminal 100. The contact confirmation unit 125 uses the determination result to output the degree of infection risk at the time of contact confirmation. The contact confirmation unit 125 may output a message indicating an appropriate action, consultation method, or the like depending on the infection risk.

As described above, in the present embodiment, in the contact confirmation system using radio signals, the reception device having reception directivity of radio waves is prepared and placed so as to correspond to the direction of the person's face or body. As a result, the received signal strength indicator varies according to the direction of the person's face or body. Therefore, it is possible to accurately evaluate the degree of contact while considering the risk of infection.

Moreover, by using the confirmation result, it is possible to determine the necessity of a close examination at a medical institution or to determine the degree of urgency.

### Second Embodiment

Hereinafter, a second embodiment will be described with reference to the drawings. The present embodiment is an example in which the information processing unit of the user terminal of the first embodiment further includes an omnidirectional beacon reception unit.

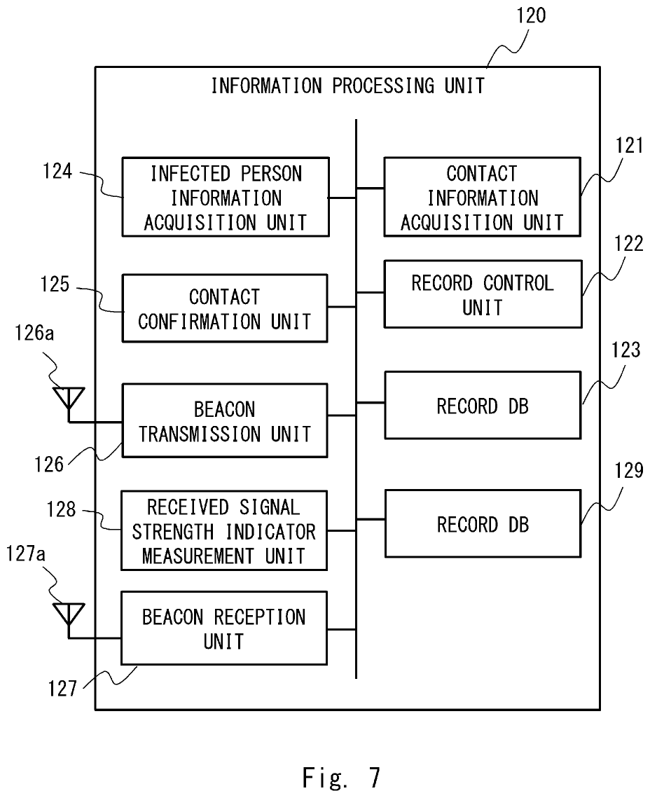

FIG. 7 illustrates a configuration example of an information processing unit 120 according to the present embodiment. As illustrated in FIG. 7, the information processing unit 120 further includes, in addition to the configuration of the first embodiment, a beacon reception unit 127, a received signal strength indicator measurement unit 128, and a record DB 129. Note that the configuration other than the information processing unit is the same as in the first embodiment.

The beacon reception unit (second beacon reception unit) 127 receives beacon signals wirelessly transmitted from a nearby user terminal 100 according to a communication standard such as BLE. The received beacon signal is the same as the beacon signal received by the reception device 110. A reception antenna 127a of the beacon reception unit 127 is an omnidirectional antenna as in the case of the beacon transmission unit 126, and has reception directivity oriented in all directions. Note that the transmission antenna 126a and the reception antenna 127a have the same characteristic, and therefore may be configured of one transmission and reception antenna.

The received signal strength indicator measurement unit (second received signal strength indicator measurement unit) 128 measures a second received signal strength indicator of the beacon signal received by the beacon reception unit 127. The record DB (second contact information recording unit) 129 records second contact information including encrypted identification information of the user of the transmission source of the beacon signal received by the beacon reception unit 127 and the second received signal strength indicator of the beacon signal measured by the received signal strength indicator measurement unit 128 under the control of a record control unit 122.

In the present embodiment, the record control unit 122 performs control such that first contact information acquired from the reception device 110 is recorded in a record DB 123 as in the first embodiment, and also the second contact information of the beacon signal received by the beacon reception unit 127 is recorded in the record DB 129. As in the case of recording of the first contact information, when the beacon reception unit 127 receives a beacon signal having the same identification information for a predetermined period, the record control unit 122 records the encrypted identification information of the user of the transmission source of the beacon signal and the second received signal strength indicator in the record DB 129 together with the received time. When the received signal strength indicator of the beacon signal transmitted from the user terminal 100 of an infected person and measured in a predetermined period (second predetermined period) recorded in the record DB 129 is a predetermined strength or greater, a contact confirmation unit 125 evaluates the degree of contact with the user terminal 100 of the infected person based on the first received signal strength indicator of the beacon signal received by the reception device 110. At this time, the contact degree is evaluated based on the total value of the first received signal strength indicator in the second predetermined period. The second predetermined period may be the same period as the first predetermined period for which the total value is obtained in the first embodiment, or may be a different period.

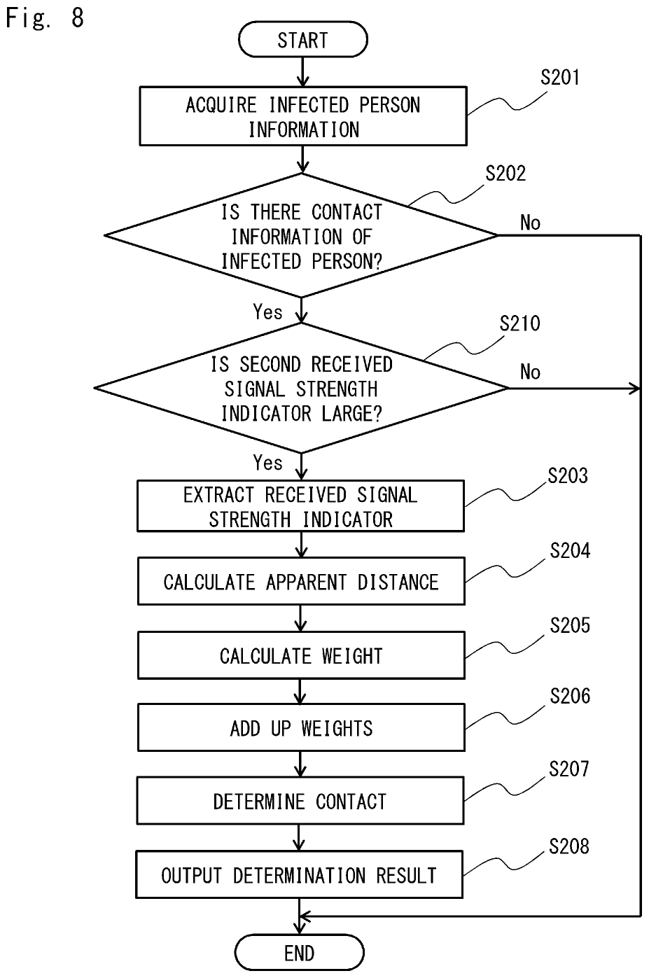

FIG. 8 illustrates an example of contact confirmation processing of the user terminal 100 according to the present embodiment. As illustrated in FIG. 8, as in the case of the first embodiment, the user terminal 100 acquires infected person information (S201), and when determining that contact information of the infected person is recorded (S202/Yes), determines whether or not the second received signal strength indicator is large (S210). The contact confirmation unit 125 refers to the record DB 129, and determines whether or not the second received signal strength indicator corresponding to the identification information of the infected person among the second received signal strength indicator of the beacon signals received by the beacon reception unit 127 is a predetermined strength or greater. If the second received signal strength indicator is smaller than the predetermined strength (S210/No), the processing is ended.

On the other hand, if the second received signal strength indicator is the predetermined strength or greater (S210/Yes), as in the first embodiment, the first received signal strength indicator is extracted (S203), contact is confirmed from the first received signal strength indicator (S204 to S207), and the determination result is output (S208).

As described above, in the user terminal of the first embodiment, an omnidirectional beacon reception unit may be further provided, and the contact confirmation of the first embodiment may be performed when the received signal strength indicator of the beacon signal received by the beacon reception unit is large. As a result, it is possible to perform the contact confirmation of the first embodiment when contact is confirmed by a function similar to a contact confirmation application. Hence, it is possible to determine contact accurately by narrowing down the confirmation target.

### Third Embodiment

Hereinafter, a third embodiment will be described with reference to the drawings. The present embodiment is an example in which the user terminal of the first or second embodiment includes a transmission and reception device instead of a reception device.

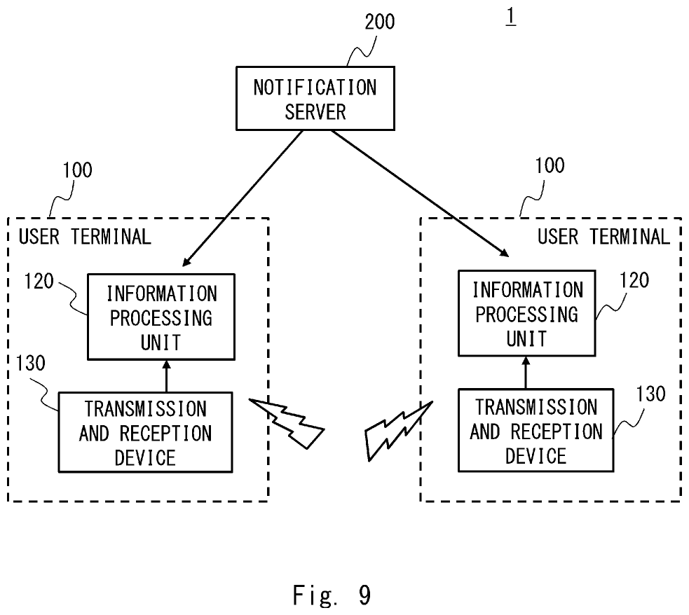

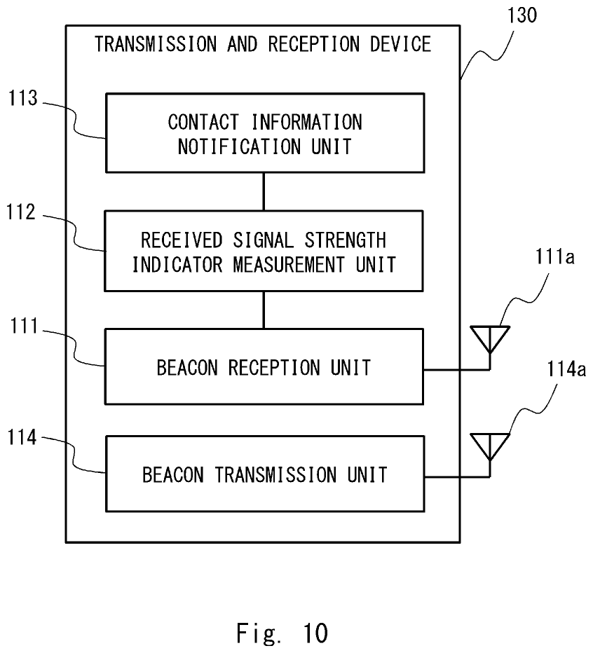

FIG. 9 illustrates a configuration example of a contact degree confirmation system according to the present embodiment. As illustrated in FIG. 9, in a contact degree confirmation system 1 according to the present embodiment, a user terminal 100 includes a transmission and reception device 130 instead of the reception device 110. The transmission and reception device 130 is not only a receiver that receives beacon signals as in the case of the reception device 110 of the first embodiment, but is also a transmitter that transmits beacon signals.

FIG. 10 illustrates a configuration example of the transmission and reception device 130 according to the present embodiment. As illustrated in FIG. 10, in addition to the configuration of the first embodiment, the transmission and reception device 130 further includes a beacon transmission unit 114. The beacon transmission unit (signal transmitter) 114 wirelessly transmits beacon signals according to a communication standard such as BLE. The transmitted beacon signal is the same as the beacon signal transmitted in the first embodiment. A transmission antenna 114a of the beacon transmission unit 114 is a directional antenna as in the case of the beacon reception unit 111, and is worn on a predetermined position on a person to have transmission directivity corresponding to the direction in which the person faces, as illustrated in FIG. 3. The reception antenna 111a and the transmission antenna 114a have the same characteristic, and therefore may be configured of one transmission and reception antenna. While both the beacon reception unit 111 and the beacon transmission unit 114 have directivity corresponding to the direction in which the person faces in this example, it is possible to assign directivity corresponding to the direction in which the person faces to only one of the two units. Note that an information processing unit 120 according to the present embodiment has a configuration in which the beacon transmission unit 126 is omitted from the configuration of the first embodiment.

As described above, in the first or second embodiment, the user terminal may include a transmission and reception device, and may have transmission directivity as well as reception directivity corresponding to the direction in which the person faces. As a result, the received signal strength indicator varies even more depending on the direction of the person on the transmission side, whereby contact can be confirmed more precisely.

Note that the present invention is not limited to the above embodiments, and changes can be made appropriately within the gist of the invention.

Each configuration of the above embodiment may be configured by hardware or software, or both, and may be configured of one hardware or software or a plurality of pieces of hardware or software. The function (processing) of each apparatus may be implemented by a computer including a CPU, a memory, and the like. For example, a storage apparatus may store a program for performing the method of the embodiment (contact information recording method and contact confirmation method), and each function may be implemented by the CPU executing the program stored in the storage apparatus.

These programs can be stored by using various types of non-transitory computer readable media and be supplied to the computer. The non-transitory computer readable medium includes various types of tangible storage media. Examples of the non-transitory computer readable medium include a magnetic recording medium (e.g., flexible disk, magnetic tape, and hard disk drive), a magneto-optical recording medium (e.g., magneto-optical disk), a CD-ROM (read only memory), a CD-R, a CD-R/W, and a semiconductor memory (e.g., mask ROM, programmable ROM (PROM), erasable PROM (EPROM), a flash ROM, and a random access memory (RAM)). Alternatively, the program may be supplied to the computer by various types of transitory computer readable media. Examples of the transitory computer readable medium include an electric signal, an optical signal, and electromagnetic waves. The transitory computer readable medium can supply the program to the computer via a wired communication path such as an electric wire, optical fiber, and the like, or a wireless communication path.

According to the present embodiment, it is possible to provide a contact degree confirmation apparatus and a contact degree confirmation system that can evaluate contact accurately.

The present invention is favorably applicable to a contact degree confirmation apparatus that confirms the degree of contact of a user.

What is claimed is:

1. A contact degree confirmation apparatus comprising:
a first signal reception unit configured to receive a radio signal including identification information of a user wearing a signal transmitter;
an infected person information acquisition unit configured to acquire identification information of a user that has caught an infectious disease that could be transmitted by droplet infection;
a first received signal strength indicator measurement unit configured to measure a first received signal strength indicator of the radio signal received by the first signal reception unit; and
a contact degree evaluation unit configured to, when identification information included in the radio signal received by the first signal reception unit coincides with identification information of the infected user, evaluate the degree of contact with the signal transmitter based on a total value of the first received signal strength indicator measured by the first received signal strength indicator measurement unit in a first predetermined period,
wherein the first signal reception unit is worn on a predetermined position of a person wearing the contact degree confirmation apparatus to have reception directivity in a front direction of a face of the person wearing the contact degree confirmation apparatus.

2. The contact degree confirmation apparatus according to claim 1, wherein the contact degree evaluation unit calculates an apparent distance from the signal transmitter based on the first received signal strength indicator, and evaluates the degree of contact based on a total value of the apparent distances with respect to a predetermined reference distance.

3. The contact degree confirmation apparatus according to claim 1, further comprising:
a second signal reception unit configured to receive a radio signal transmitted by the signal transmitter; and
a second received signal strength indicator measurement unit configured to measure a second received signal strength indicator of the radio signal received by the second signal reception unit,
wherein when identification information included in the radio signal received by the second signal reception unit coincides with identification information of the infected user and the second received signal strength indicator measured by the second received signal strength indicator measurement unit in a second predetermined period is a predetermined strength or greater, the contact degree evaluation unit evaluates the degree of contact based on the first received signal strength indicator.

4. The contact degree confirmation apparatus according to claim 3, wherein the contact degree evaluation unit evaluates the degree of contact based on a total value of the first received signal strength indicator in the second predetermined period.

5. A contact degree confirmation system comprising:
a signal transmitter; and
a contact degree confirmation apparatus,
wherein:
the signal transmitter includes a signal transmission unit that transmits a radio signal including identification information of a user wearing the signal transmitter;
the contact degree confirmation apparatus includes
a first signal reception unit that receives a radio signal transmitted by the signal transmitter,
an infected person information acquisition unit that acquires identification information of a user that has caught an infectious disease that could be transmitted by droplet infection,
a first received signal strength indicator measurement unit that measures a first received signal strength indicator of the radio signal received by the first signal reception unit, and
a contact degree evaluation unit that, when identification information included in the radio signal received by the first signal reception unit coincides with identification information of the infected user, evaluates the degree of contact with the signal transmitter based on a total value of the first received signal strength indicator measured by the first received signal strength indicator measurement unit in a first predetermined period; and
the first signal reception unit is worn on a predetermined position of a person wearing the contact degree confirmation apparatus to have reception directivity in a front direction of a face of the person wearing the contact degree confirmation apparatus.
